(19) United States
(12) Patent Application Publication
(10) Pub. No.: US 2008/0142030 A1
(43) Pub. Date: Jun. 19, 2008

(54) Hair dye touch-up dispenser and method of using the same

(75) Inventors: Marc A. Venere, Maple (CA); Robert G. Dickie, Newmarket (CA)

Correspondence Address: SAND & SEBOLT, AEGIS TOWER, SUITE 1100, 4940 MUNSON STREET, NW, CANTON, OH, 44718-3615, US

(73) Assignee: MARC ANTHONY VENTURE CORPORATION, Concord (CA)

(21) Appl. No.: 11/638,171

(22) Filed: Dec. 13, 2006

(51) Int. Cl. A45D 24/22; A61K 8/18; B67D 5/56; B65D 25/08

(52) U.S. Cl. 132/116; 222/129; 132/208; 206/219

(57) ABSTRACT

A two-container dispenser for storing, mixing and dispensing hair dye for touch up applications. The dispenser retains and separates two hair dye components until they are to be mixed immediately prior to application of the dye. The two containers are permanently secured together via a valve assembly. When the valve assembly is in a closed position, the interior chambers and therefore the contents of the containers remain separated. When the valve assembly is opened by rotating one of the containers through 180 degrees, the two interior chambers are in fluid communication and the contents can flow between the containers to form a hair dye mixture. The connections between the first and second containers and the valve assembly are such that all three remain secured together when the valve assembly is moved between the closed and open positions.

### BACKGROUND OF THE INVENTION

1. Technical Field

This invention generally relates to hair care products. More particularly, the invention relates to hair dyes. Specifically, the invention relates to a hair dye dispenser that comprises two chambers that are permanently secured to each other via a valve assembly and which permit liquids stored separately in the two chambers to be mixed prior to dispensing by simply rotating one of the chambers relative to the other.

2. Background Information

It has become commonplace for people to apply dye to their hair to completely change the color of the hair or to apply highlights thereto in order to improve their overall appearance. This alteration in the color may be done in a salon by trained professionals or by the individual in the comfort of their own home.

There are a variety of hair coloring systems on the market and the product selected depends on how permanent the color change is to be. Hair products may be semi-permanent, demi-permanent or permanent in nature. Semi-permanent products contain no ammonia and coat the hair with a color. The color tends to wash out of the hair over six to twelve shampoos. Demi-permanent products also contain no ammonia but do contain some peroxide. Consequently, they produce more dramatic color changes than the semi-permanent products and also tend to last longer than semi-permanent products. The demi-permanent dyes typically last between twenty and twenty-five shampoos. Finally, permanent products are used to permanently change the color of the hair or may be applied to only some strands of hair to create highlights. Permanent products permanently change the color of that portion of the hair shaft that is contacted by the product. As such, they cannot be washed out of the hair, the color change has to be “grown out” of the hair. People who have used permanent products on their hair are typically concerned about their roots—, i.e., any new hair that grows after the application of the hair product will be the natural color of the person's hair. That natural hair color differs from the color of the dyed hair and the overall effect may be aesthetically displeasing. Furthermore, activities such as swimming in chlorinated pools can strip, fade and change the color of the hair. Consequently, the hair has to be dyed again every four to six weeks. However, in the interim, it may be desirable to touch up the hair color and reduce the difference between the roots and the dyed hair or to rejuvenate the color. This may be accomplished through the use of a touch-up coloring system.

All permanent hair dyes are created by mixing together a solution of a colorant dye that includes ammonia with a developer solution of hydrogen peroxide. Touch-up systems therefore tend to come with a bottle of ammonia-containing colorant and a separate bottle of hydrogen peroxide solution. A small open tray is provided for the mixing of these two chemical solutions. These systems are prone to user spillage and both the dye solution and the peroxide can permanently stain and damage furniture and clothing. Additionally, spilling even a small amount of either liquid will alter the end color of the dye and will therefore affect the outcome. Furthermore, the actual act of applying the dye to the hair by an untrained individual can be a messy and inexact process.

Several devices have been proposed in the prior art to address these issues. These include the devices disclosed in U.S. Pat. No. 4,823,946 issued to Stoeffler et al; U.S. Pat. No. 5,277,303 issued to Goyet et al; U.S. Pat. No. 5,647,481 issued to Hundertmark et al and U.S. Pat. No. 7,000,618 issued to Dovergne et al. All of these patents disclose containers that have two separated chambers that store liquids separately and allow them to be mixed immediately prior to application of the same. However, in each device at least one, and sometimes both, of the chambers must be detached from the container, opened and then reconnected in some manner to the other chamber for mixing prior to dispensing of the mixture. While this makes for neat packaging of the liquids and easier mixing of the same, it does not do away with the problems created by the potential for spillage of some of the liquid when the chambers are opened and reconnected.

There is therefore a need in the art for an improved touch up dispenser for hair dye.

### SUMMARY OF THE INVENTION

The device of the present invention comprises a two-container dispenser that is adapted to retain and separate two hair dye components until they are to be mixed immediately prior to application of the dye. The dispenser includes a mechanism for allowing transfer of the contents of one container into the other without having to detach either container from the dispenser. The dispenser further allows for mixing of the contents of the two containers and for dispensing the mixed dye from the container by simply rotating one of the containers relative to the other. The dispenser comprises two containers that are substantially permanently connected together via a valve assembly. The dispenser further includes an applicator that is secured to one of the containers and includes a dispensing hole through which the mixture is dispensed. The applicator further includes a plurality of bristles onto which the dispensed dye is fed. A cap is provided to close the dispensing hole and to cover and protect the bristles until the device is in use. The valve assembly is movable between a closed position and an open position by rotating one of the containers through an angle that is sufficient to open the valve assembly. In the preferred embodiment of the invention, second container is rotated through 180 degrees in a counter-clockwise direction. When the valve assembly is in an open position, the two components can be transferred from one container to the other. The dispenser can be shaken to ensure proper mixing of the components without fear of spillage or leakage from the dispenser. The connections between the first and second containers and the valve assembly are such that all three remain secured together when the valve assembly is moved from a closed position to an open position. Furthermore, the rotation of the containers relative to each other and to the valve assembly tears a perforated tamper evident label that is secured about the dispenser. This serves as an indicator to the consumer that the dispenser has been previously handled and that any mixed dye contained therein may have become ineffective.

### BRIEF DESCRIPTION OF THE DRAWINGS

The preferred embodiments of the invention, illustrative of the best mode in which applicant has contemplated applying the principles, are set forth in the following description and are shown in the drawings and are particularly and distinctly pointed out and set forth in the appended claims.

FIG. 1 is a perspective view of a hair dye dispenser in accordance with the present invention and showing the cap removed therefrom;

FIG. 2 is an exploded perspective view of the dispenser of FIG. 1;

FIG. 2a is a top view of the valve body;

FIG. 3 is a front view of the hair dye dispenser;

FIG. 4 is a cross-sectional front view of the dispenser through line A-A of FIG. 3 and showing the dispenser in a closed non-dispensing position;

FIG. 5 is a cross-sectional side view of the dispenser through line B-B of FIG. 4;

FIG. 6 is a cross-sectional front view of the dispenser in an open position where the contents of the chambers can be mixed; and

FIG. 7 is a cross-sectional top view of the dispenser through line C-C of FIG. 6.

### DETAILED DESCRIPTION OF THE INVENTION

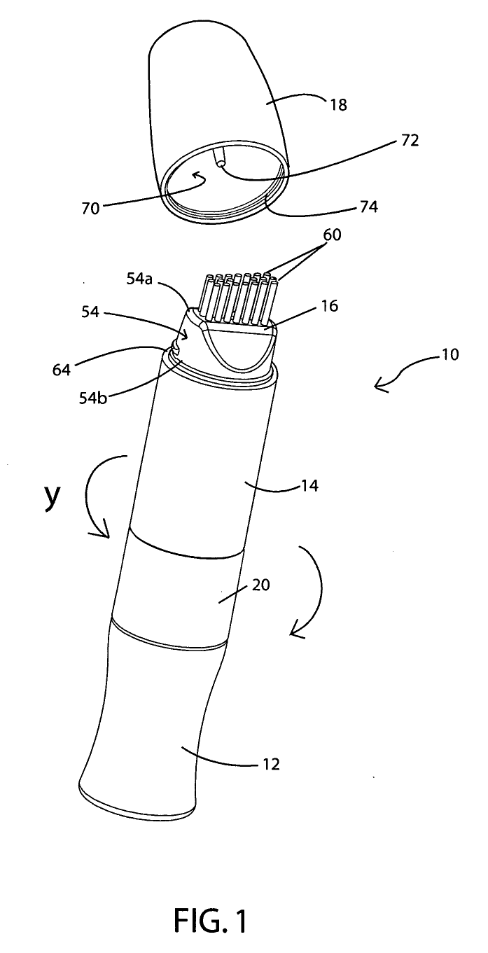

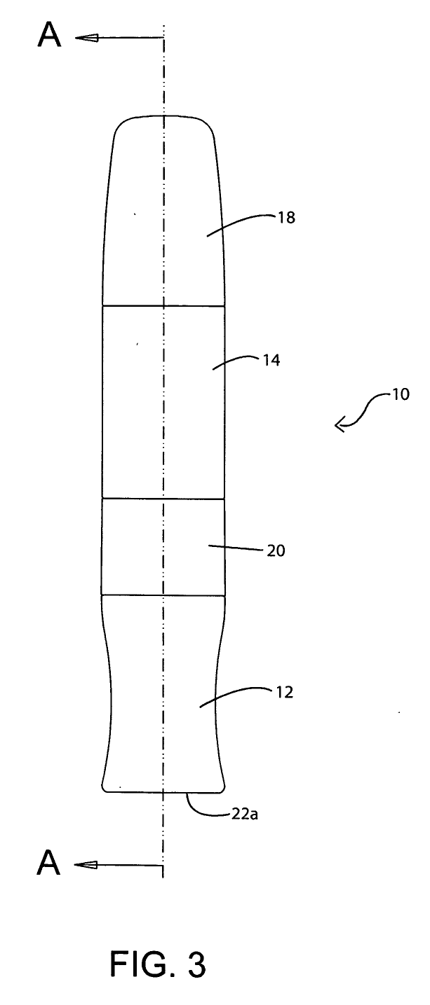

Referring to FIGS. 1-7, there is shown a hair dye dispenser in accordance with the present invention and generally indicated at 10. Dispenser 10 comprises a first container 12, a second container 14, an applicator 16, and a cap 18. A valve assembly 20 is provided to permanently connect first and second containers 12, 14 together. The first container 12 is adapted to retain a first component (not shown) of the hair dye therein. The second container 14 is adapted to retain a second component (not shown) of the hair dye therein. The first and second containers 12, 14, the applicator 18 and valve assembly 20 are permanently fixed together during the stages of separate storage of the first and second components of the hair dye, mixing of the first and second components; and dispensing of the mixed first and second components. At no time are any of the first and second containers 12, 14, the applicator 18 and valve assembly 20 detached from each other to perform any of these steps.

First container 12 comprises a generally cylindrical body 22 that has a bottom wall 22a, a cylindrical side wall 22b extending outwardly away from bottom wall 22a, and an upper end 22c remote from bottom wall 22a. Upper end 22c is circumferentially narrower than side wall 22b. Body 22 defines an interior chamber 24 that is adapted to hold the first component (not shown) of the hair dye. Typically, that first component is in liquid form. So, for example, interior chamber 24 may retain a hydrogen peroxide solution therein. The exterior surface of upper end 22c is provided with first threads 26 and includes an opening 28 that provides access to interior chamber 24. First threads 26 preferably are oriented in such a manner that they are counterclockwise.

Second container 14 also comprises a generally cylindrical body 28. Body 28 has a side wall 28a that surrounds an interior chamber 30. Body 28 also has an upper end 28b and a lower end 28c, both of which are circumferentially narrower than side wall 28a. In the preferred embodiment of the invention, lower end 28c is substantially the same size as upper end 22c of first container 12. Upper end 28b of second container 14 is smaller than both of upper end 28b and lower end 28c. The external surface of upper end 28b is provided with second threads 32 and has a first opening 34 therein that provides access to interior chamber 30. The external surface of lower end 28c is provided with third threads 35. Third threads 35 terminate in a slightly enlarged latch 37. The purpose of latch 37 will be described later in this specification. Lower end 28c includes a second opening 36 into interior chamber 30. Interior chamber 30 is adapted to hold the second component (not shown) of the hair dye. Again, second component is typically a liquid, such as a liquid colorant dye, but could also be a solid material.

In accordance with a specific feature of the present invention, valve assembly 20 is disposed between and permanently connected to both of the first and second containers 12, 14. Valve assembly 20 comprises a substantially rigid valve body 38, a flexible and resiliently compressible gasket 40, and a rigid valve seat 42. Valve body 38 and valve seat 42 preferably are manufactured from a substantially rigid plastic material.

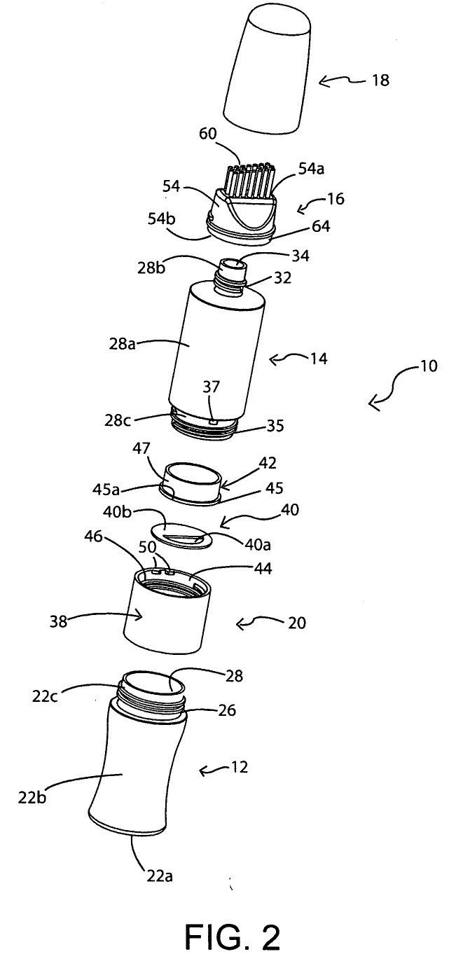

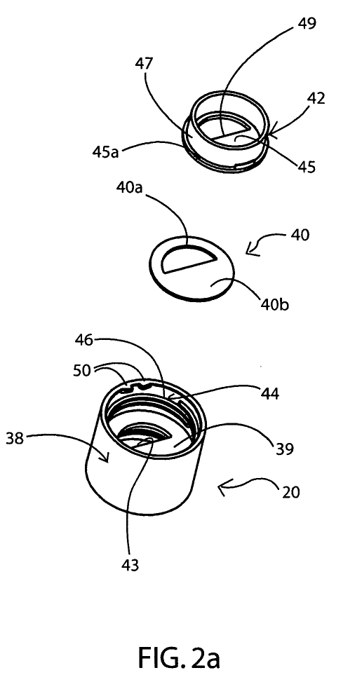

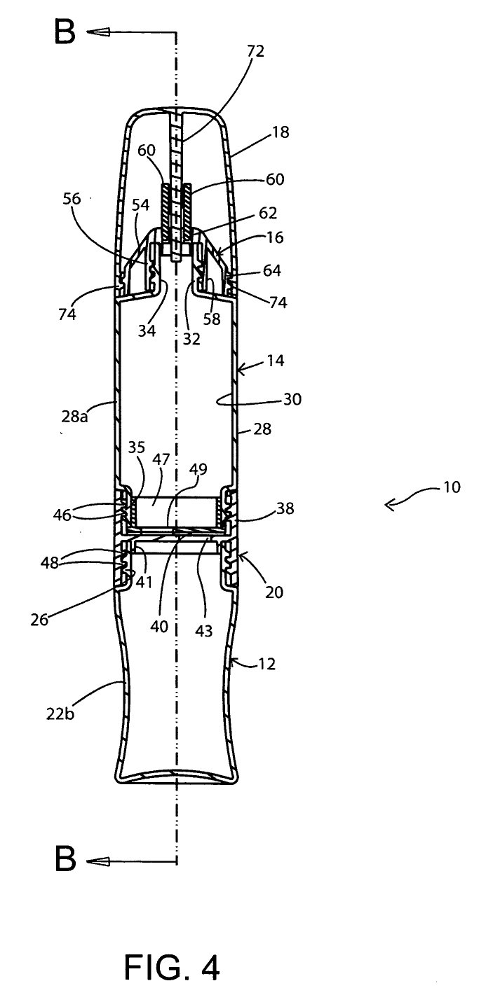

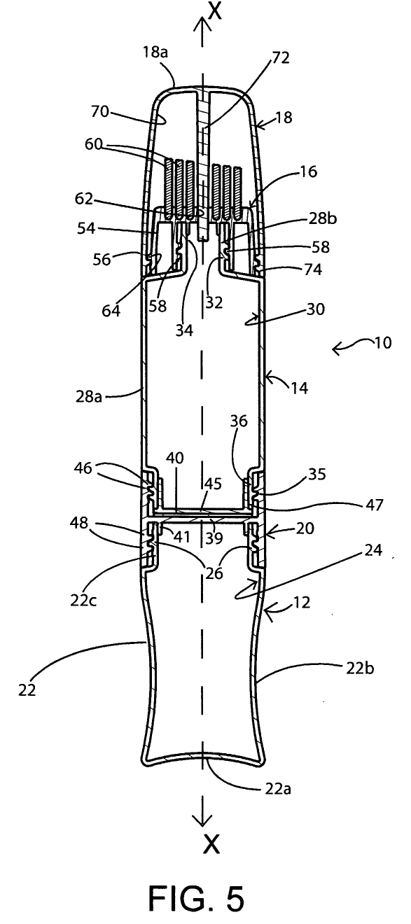

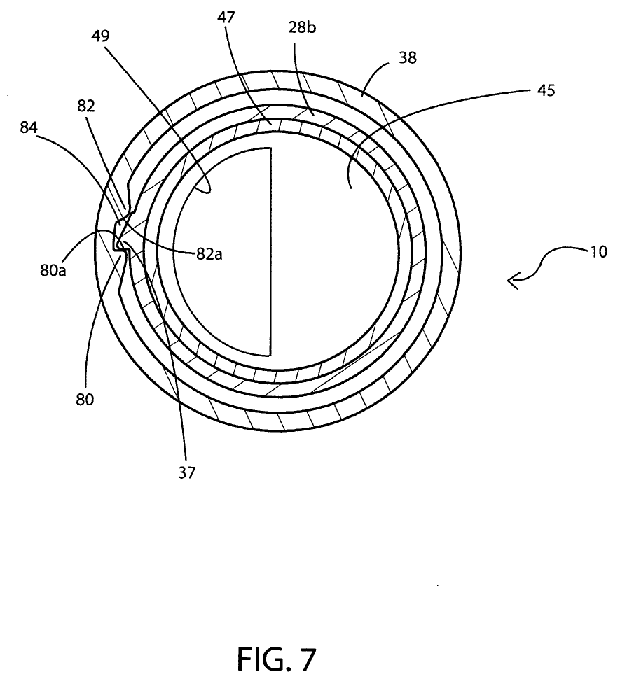

In accordance with a further specific feature of the present invention, valve body 38 is generally tubular in shape and has an external diameter that is substantially the same as the external diameters of side walls 22b, 28a so that dispenser 10 appears to be a substantially continuous container. Valve body 38 has a central bore 44 that is sized to receive the upper end 22c of first container 12 and the lower end 28c of second container 14 therein. Valve body 38 is provided with an interior wall 39 that extends across bore 44 and substantially at a right angle to the longitudinal axis “X” (FIG. 5) of dispenser 10. Interior wall 39 includes an annular lip 41 that extends outwardly away therefrom and is sized to be tightly received within opening 28 when valve assembly 20 is engaged with first container 12. Interior wall 39 is perforated by a D-shaped aperture 43 (FIG. 2a). The interior surface of valve body 38 is provided with fourth and fifth threads 46, 48 (FIG. 4) that are spaced apart from each other. Fourth threads 46 are positioned to engage third threads 35 on lower end 28c of second container 28. Fifth threads 48 are positioned to engage first threads 26 on upper end 22c of first container. Interior wall 39 is disposed intermediate fourth and fifth threads 46, 48. The interior surface of valve body 38 further includes a rotation stop mechanism 50 disposed outwardly of fourth threads 46. Stop mechanism 50 is shown in greater detail in FIG. 7. Mechanism 50 comprises two spaced-apart detents 80, 82 which are separated from each other by a groove 84. Detent 80 is shaped so as to present an inner wall 80a that is oriented at right angles to the direction of counterclockwise rotation of valve body 38 around longitudinal axis “X”. Inner wall 82a of detent 82 is slanted at an angle other than ninety degrees.

Gasket 40 is manufactured from a flexible, deformable material and includes a D-shaped aperture 40a therein. An adhesive layer (not shown) is applied to upper surface 40 of gasket 40 so that it may be secured to valve seat 42.

Valve seat 42 has a bottom wall 45 that is sized to be received within bore 44 of valve body 38. A peripheral wall 47 extends upwardly away from bottom wall 45 a small spaced distance inwardly from the outer edge thereof. Consequently, a small lip 45a extends annularly outwardly away from peripheral wall 47. Peripheral wall 47 is positioned to extend through the second opening 36 in the lower end 28c when valve assembly 20 is engaged with second container 14. When peripheral wall 47 is so received, it tightly abuts the interior surface of the lower end 28c as shown in FIG. 4. Bottom wall 45 includes a D-shaped aperture 49 that is configured to be substantially identical to the D-shaped apertures 40a and 43 in gasket 40 and valve body 38, respectively.

Referring to FIGS. 4-6, applicator 16 comprises a body that has an exterior wall 54 and an interior wall 56 that extend downwardly away from an interior surface of exterior wall 54. Interior wall 56 is substantially cylindrical in shape and has an opening 57 of a diameter sized to receive upper end 28b of second container 14 therein. The interior surface of interior wall 56 is provided with sixth threads 58 that are positioned to engage second threads 32 on upper end 28b of second container 14. Sixth threads 58 are oriented in a counterclockwise direction.

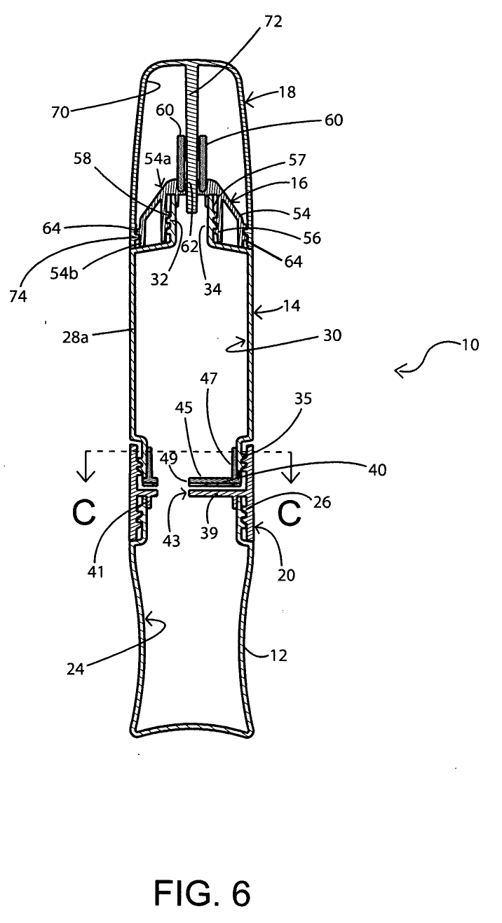

Exterior wall 54 of applicator 16 has a narrower upper end 54a and a wider, lower end 54b. The diameter of lower end 54b is substantially the same as the external diameter of side walls 22b, 28a of first and second containers 12, 14. The external surface of the lower end 54b of exterior wall 54 is provided with seventh threads 64. A plurality of plastic bristles 60 are secured to and extend outwardly away from upper end 54a. A small dispensing hole 62 (FIG. 6) is provided in upper end 54a in the midst of bristles 60.

Cap 18 is designed to detachably be received over applicator 16. Cap 18 comprises a body that defines a hollow interior chamber 70. A tapered pin 72 extends downwardly from the interior surface of the top end 18a of cap 18. When cap 18 is attached to dispenser 10, pin 72 preferably is disposed substantially along the longitudinal axis “X”. Pin 72 is positioned to be received through hole 62 in upper end 54a of applicator 16 and is sized to plug the same when so received. The interior surface of cap 18 is also provided with eighth threads 74 that are positioned to engage seventh threads 64 on applicator 16.

Dispenser 10 is assembled in the following manner. Valve body 38 is positioned over upper end 22c of first container 12 so that fifth threads 48 on valve body 38 threadably engage first threads 26 on first container 12. First container 12 is at least partially filled with the first liquid component (not shown) of the hair dye, such as a hydrogen peroxide solution. Gasket 40 is adhesively secured to the bottom wall 45 of valve seat 42 in such a manner that D-shaped apertures 40a and 49 are aligned with each other. Valve seat 42 and gasket 40 thereby act as a single unit. Valve seat 42 and gasket 40 are then engaged with lower end 28c of second container 14. Peripheral wall 47 of valve seat 42 is inserted into second opening 36 in lower end 28c and because the circumference of peripheral wall 47 closely approximates the diameter of second opening 36, valve seat 42 and gasket 40 become frictionally engaged with second container 14. Consequently, when second container 14 is rotated, valve seat 42 and gasket 40 travel with it.

Lower end 28c of second container 14, with engaged valve seat 42 and gasket 40, is then inserted into central bore 44 of valve body 38. Second container 14 is secured to valve body 38 by threadably engaging third threads 35 with fourth threads 46. Third and fourth threads 35, 46 are oriented in such a manner that when they are engaged, the first and fifth threads 26, 48 do not disengage, but instead become further tightened. Second container 14 is rotated to the point that gasket 40 occludes the D-shaped aperture in valve body 38 (FIG. 4.), i.e., to the point that the valve assembly 20 is in the closed position. At this point, the interior chamber 24 of first container 12 is sealed by gasket 40 and is also simultaneously separated from the interior chamber 30 of second container 14. Second container 14 is then at least partially filled, through first opening 34, with a second dye component such as a liquid colorant dye (not shown).

The applicator 16 is then placed over upper end 28b of second container 14 so that upper end 28b is received into opening 57 (FIG. 6) in interior wall 56. Second threads 32 on upper end 28b are threadably engaged with sixth threads 58 on interior wall 56. Cap 18 is positioned over applicator 16 so that pin 72 is received through hole 62. Seventh threads 64 on applicator 16 are threadably engaged with eighth threads 74 on cap 18. Pin 72 effectively seals second container 14 and prevents any the second component of the hair dye from leaking out of said second container 14.

A product label (not shown) preferably is then applied around the exterior surface of at least a portion of the connected first container 12, valve assembly 20, second container 14, and at least a portion of cap 18. The label preferably is provided with perforations and directions that indicate to the consumer where and how to rotate the relative component parts of dispenser 10. More specifically, the label will indicate firstly how to rotate the second container 14 so as to open the valve assembly 20 and thereby permit mixing of the first and second liquid components that have been previously separated. The label will secondly indicate how to rotate cap 18 so as to remove it from over applicator 16 and to thereby open hole 62 for dispensing of mixed hair dye therethrough. Because the label rips when the components are rotated relative to each other, it therefore also provides a tamper-evident indicator for the consumer.

The dispenser 10 is used in the following manner. When purchased by the consumer, the valve assembly 20 is in the closed position shown in FIGS. 4&5. This means that apertures 49, 40a in the combined valve seat 42 and gasket 40 are out of alignment with aperture 43 in valve body 38. Consequently, gasket 40 separates interior chambers 24, 30 from each other and thereby is keeping the first and second components of the hair dye separated from each other. Furthermore, because cap 18 is engaged with applicator 16, the pin 72 is tightly seated in hole 62 and the contents of second container 14 are retained therein.

When the consumer wishes to dye their hair, they grasp dispenser 10 and rotate the second chamber 14 through 180 degrees in a counterclockwise direction relative to valve assembly 20 and first container 12. This is indicated by the arrow “Y” in FIG. 1. This counterclockwise rotation through 180 degrees brings the D-shaped apertures 49, 40a in valve seat 42 and gasket 40, into alignment with D-shaped aperture 43 in valve body 38. FIGS. 2&7 show that dispenser 10 is provided with the stop mechanism 50 that ensures that there is no over-rotation of the second container 14. Stop mechanism 50 also provides an indicator to the consumer that there is exact alignment of the D-shaped apertures 43, 49 and 40a and the valve assembly 20 is in the fully open position and first and second interior cavities 24, 30 are in fluid communication with each other. Stop mechanism 50 functions as follows. As mentioned previously, the terminal end of the third threads 35 on the lower end 28c of second container 28 is slightly enlarged to form a latch 37. As second container 14 is rotated, latch 37 slides over detent 82 and into groove 84. First and second containers 12, 14 are blow-molded and are flexible in nature. Consequently, latch 37 is able to slide over detent 82 and into groove 84 when the second container is moved in a counter-clockwise direction. Once in groove 84, latch 37 abuts right angled inner wall 80a of detent 80 and no further rotation of the second container 14 in a counter-clockwise direction is possible. Inner wall 82a of detent 82 is slanted at an angle other than ninety degrees but still provides sufficient resistance to latch 37 to maintain it within groove 84. Valve assembly 20 is therefore retained in fully open position unless a force is applied to rotate second container 14 in a clockwise direction. It should be noted that the fifth threads 48 on valve body 38 that engage first threads 26 on first container 12 are oriented so that the counter-clockwise rotation of second container 14 tightens the connection between valve body 38 and first container 12. It will be understood that because of the arrangement and functioning of the various elements of dispenser 50, that dispenser 50 grows slightly in overall length when second container 14 is rotated in a counter-clockwise direction. The resiliency of gasket 40 allows it to expand slightly to occupy the space between valve seat 42 and valve body 38 so that no liquids leak from dispenser 10.

Dispenser 10 is now in a condition where the contents of interior chambers 24, 30 of first and second containers 12, 14 can flow in either direction through the aligned apertures 49, 43 and 40a. The consumer then shakes dispenser 10 vigorously to mix the first and second hair dye components. Directions printed on the label will instruct the consumer how long to shake dispenser 10 to ensure proper mixing of the first and second components. Once dispenser 10 has been shaken for the required amount of time, the consumer grasps dispenser 10 in such a manner as to rotate cap 18 in a counter-clockwise direction. This rotation causes eighth threads 74 to disengage from seventh threads 64, releasing the cap 18 from applicator 16. As cap 18 is removed from applicator 16, pin 72 is withdrawn from hole 62. The dispenser 10 may then be inverted and mixed hair dye flows out of hole 62 and onto bristles 60. The dye may thereby be transferred onto strands of the consumer's hair as the bristles 60 are engaged therewith.

The cap 18 may be threadably engaged with applicator 16 to seal off second container 14 and prevent any further dispensing of mixed dye therefrom.

It has been disclosed, in this specification, that first and second threads 26, 32 are oriented in a counter-clockwise direction and that rotating second container 14 and cap 18 in a counter-clockwise direction simply tightens the engagement of first container 12 with valve body 38 and applicator 16 with second container 14. It will, however, be understood that first and second threads 26, 32 could instead be oriented in a clockwise direction and second container 14 and cap 18 could be rotated in a clockwise relative to valve body 20 and applicator 16 respectively without departing from the spirit of the present invention, although this arrangement of components would not be conventional.

It will also be understood that while stop mechanism 50 is provided on valve body 38 and latch 37 is provided on second container 14; the stop mechanism could, alternatively, be provided on the second container 14 and the latch provided on the valve body 38 without departing from the spirit of the present invention.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention are an example and the invention is not limited to the exact details shown or described.

1. A dispenser for holding, mixing and dispensing hair dye, said dispenser comprising:
a first container having a first interior chamber that is adapted to retain a first component of the hair dye therein;
a second container having a second interior chamber that is adapted to retain a second component of the hair dye therein;
a valve assembly permanently connected to the first and second containers; said valve assembly being movable between a closed position, where the first and second interior chambers are separated from each other; and an open position where the first and second interior chambers are in fluid communication with each other; and wherein the valve assembly is movable between the open and closed positions without separating either of the first and second containers from the valve assembly.

2. The dispenser as defined in claim 1; wherein the valve assembly comprises:
a valve body having:
a first end;
a second end remote from the first end;
a bore extending between the first and second ends;
a transverse wall disposed within the bore and extending across the same substantially at a right angle thereto;
a first aperture defined within the transverse wall; and
a valve sleeve receivable within the bore such that the valve sleeve is disposed between the transverse wall and the second end of the valve body; and wherein said valve sleeve defines a second aperture therein; and wherein at least one of the valve body and valve sleeve are rotatable between a first position where the first and second apertures are non-aligned and the valve assembly is in the closed position; and a second position where the first and second apertures are aligned and the valve assembly is in the open position.

3. The dispenser as defined in claim 2, wherein each of the first and second apertures are substantially D-shaped.

4. The dispenser as defined in claim 2, wherein the valve assembly further comprises a gasket; said gasket being disposed between the valve sleeve and the transverse wall.

5. The dispenser as defined in claim 4, wherein the gasket defines a third aperture; and said third aperture is disposed so as to be non-aligned with at least one of the first and second apertures when the valve assembly is in the closed position; and is aligned with both of the first and second apertures when the valve assembly is in the open position.

6. The dispenser as defined in claim 2 wherein:
the first container has an upper end that is provided with a plurality of externally disposed first threads;
the second container has an upper end that is provided with a plurality of externally disposed second threads and a lower end that is provided with a plurality of externally disposed third threads;
the first end of the valve body is provided with a plurality of internally disposed fourth threads; and
the second end of the valve body is provided with a plurality of internally disposed fifth threads; and wherein the first and fifth threads are threadably engaged to connect the first container to the valve body, and the third and fourth threads are threadably engaged to connect the second container to the valve body.

7. The dispenser as defined in claim 6, wherein the first and second threads are oriented in a substantially counter-clockwise direction.

8. The dispenser as defined claim 2, wherein the second container is rotatable through 180 degrees in a counter-clockwise direction relative to the first container to move the valve assembly from the closed position to the open position.

9. The dispenser as defined in claim 8, wherein one of the valve body and second container includes a stop mechanism and the other of the valve body and second container includes a latch that is engageable with the stop mechanism to stop the rotation of the second container relative to the valve body.

10. The dispenser as defined in claim 9, wherein the stop mechanism comprises:
a pair of spaced apart detents formed on an interior surface of the valve body that defines the bore; the detents of the pair of detents being separated from each other by a groove which is oriented at 90 degrees to the direction of counter-clockwise rotation; and the latch is engageable in the groove.

11. The dispenser as defined in claim 10, wherein the latch comprises a terminal end of the third threads.

12. The dispenser as defined in claim 11, wherein a first of the detents includes a right angled inner wall that prevents the latch from moving past it as the second container is rotated, thereby arresting the movement of the second container.

13. The dispenser as defined in claim 6, further comprising an applicator that is engageable with the second container; whereby the second container is disposed between the applicator and the valve assembly.

14. The dispenser as defined in claim 13, wherein the applicator comprises:
a body having an inner end and an outer end;
a plurality of internally disposed sixth threads formed on an interior surface of the inner end of the body; said sixth threads being threadably engageable with the second threads on the second container;
a plurality of externally disposed seventh threads formed on an exterior surface of the inner end of the body; and
a plurality of bristles extending outwardly away from the outer end of the body.

15. The dispenser as defined in claim 14, wherein the outer end of the applicator body defines a dispensing hole that is disposed among the bristles; and wherein the dispensing hole is in fluid communication with the second interior chamber when the applicator and second container are engaged with each other.

16. The dispenser as defined in claim 15, further comprising a cap that is selectively receivable over the applicator; and wherein said cap has an interior surface that is provided with a plurality of eighth threads that are selectively engageable with the seventh threads on the applicator.

17. A method of storing, mixing and dispensing permanent hair dye for touch-up applications using a single dispenser; said method comprising the steps of:
storing a first component of the hair dye in a first interior cavity of a first container of the dispenser;
storing a second component of the hair dye in a second interior cavity of a second container of the dispenser; wherein the second container is permanently linked to the first container via a valve assembly; and when the valve assembly is in a closed position, the first interior cavity is separated from the second interior cavity and the first component is therefore separated from the second component;
rotating the second container through an angle to move the valve assembly from a closed position to an open position where the first and second interior cavities are in fluid communication with each other;
shaking the dispenser to move the first and second components between the first and second interior cavities and to thereby form a hair dye mixture;
inverting the dispenser to allow the hair dye mixture to flow through a hole in an applicator that is in fluid communication with the second interior cavity of the second container; and
applying the hair dye mixture to the hair.

18. The method as defined in claim 17, wherein the step of rotating the second container includes rotating the second container through an angle of 180 degrees in a counter-clockwise direction to move the valve assembly from an open position to a closed position.

19. The method as defined in claim 17, further comprising the step of:
rotating a cap that is threadably engaged with the applicator so as remove a pin from the hole and to thereby allow the dye mixture to flow therethrough.

20. The method as defined in claim 19, wherein the step of rotating the cap comprises rotating the cap through an angle of 180 degree in a counter-clockwise direction.
